(19) United States
(12) Patent Application Publication
(10) Pub. No.: US 2026/0185933 A1
(43) Pub. Date: Jul. 2, 2026

(54) APPARATUS FOR INSPECTING COMPONENT AND METHOD OF INSPECTING COMPONENT

(71) Applicant: SEMES CO., LTD., Cheonan-si (KR)

(72) Inventors: Young Ran KO, Suwon-si (KR); Chang Jin MOON, Seoul (KR); Ga Yeon KIM, Hwaseong-si (KR); Hong Moon KIM, Cheonan-si (KR); Hyeon Ho LEE, Hwaseong-si (KR); Soo Woong KIM, Yongin-si (KR); Tae Ho KANG, Suwon-si (KR)

(73) Assignee: SEMES CO., LTD., Cheonan-si (KR)

(21) Appl. No.: 19/432,332

(22) Filed: Dec. 24, 2025

(30) Foreign Application Priority Data
Dec. 27, 2024 (KR) 10-2024-0198537

(51) Int. Cl. G01N 21/3563; G01N 21/95; G01N 33/44

(52) U.S. Cl. CPC G01N 21/3563; G01N 21/95; G01N 33/442; G01N 2201/021

(57) ABSTRACT

Disclosed are a component inspection apparatus and a component inspection method that inspect a component in a non-contact and non-destructive manner. According to an exemplary embodiment of the present invention, an emitting unit emits light to a component, a light receiving unit receives transmitted or reflected light from the component, and a controller acquires information from the received light to recognize a fraction of fillers in the component. The above process may be repeatedly and continuously performed while moving the component, and accordingly, the degree of dispersion of the filler in the component may be predicted.

### CROSS-REFERENCE TO RELATED APPLICATION

This application claims priority to and the benefit of Korean Patent Application No. 10-2024-0198537 filed in the Korean Intellectual Property Office on Dec. 27, 2024, the entire contents of which are incorporated herein by reference.

### TECHNICAL FIELD

The present invention relates to a component inspection apparatus and a component inspection method, and more specifically, to a component inspection apparatus and a component inspection method that are capable of quickly inspecting a component in a non-contact and non-destructive manner.

### BACKGROUND ART

Polymer and plastic-based materials are widely used in various industries due to their advantages, such as lightweight, processability, and economic feasibility, but their use is often limited in certain applications due to low temperature of use, limited physical properties and strength, and performance limitations.

In order to overcome this limitation, a method of introducing various additives or fillers (hereinafter, referred to as “filler”) into a polymer matrix has been used. By adding a ceramic or metal filler to the polymer matrix, the properties of the material may be greatly improved, thereby obtaining superior physical properties and performance than pure polymer materials.

However, in the process of adding the filler, the filler may be unevenly dispersed. This may cause material non-uniformity, which may cause problems, such as deviation of physical properties according to the area of the component, the increase of the possibility of local damage, and risk of overall quality degradation. Therefore, in order to ensure the quality of composite material components, thorough analysis and management are required from the manufacturing stage to verify whether the filler is properly dispersed. To this end, a non-destructive inspection method is required, and a rapid measurement method is required for process efficiency.

### SUMMARY OF THE INVENTION

The present invention has been made in an effort to provide a component inspection apparatus and a component inspection method capable of inspecting the distribution of fillers F in a component by a non-destructive inspection method.

The present invention has been made in an effort to provide a component inspection apparatus and a component inspection method capable of checking the degree of dispersion of the fillers F from the manufacturing stage.

The objectives of the present disclosure are not limited thereto and other objectives not stated herein may be clearly understood by those skilled in the art from the following description.

An exemplary embodiment of the present disclosure, an apparatus for inspecting a component, the apparatus comprising: a stage for supporting a component; an emitting unit configured to emit light to the component supported on the stage; a light receiving unit configured to receive light from the component supported on the stage; and a controller, and wherein the controller predicts a fraction of fillers in the component through information about the light received by the light receiving unit, the information includes: a time taken to receive the light emitted by the emitting unit; a difference between an intensity of light emitted in the emitting unit and an intensity of light received in the light receiving unit; and a degree of refraction of the light emitted in the emitting unit.

Further, An exemplary embodiment of the present disclosure, a method of inspecting a component, the method comprising: an emitting operation of emitting light to the component; a light receiving operation of receiving the light from the component; and a predicting operation of predicting a fraction of fillers in the component through information about the received light, wherein the information may include: a time taken to receive the light emitted in the emitting operation; a difference between an intensity of light emitted in the emitting operation and an intensity of light received in the light receiving operation; and a degree of refraction of the light emitted in the emitting operation.

Further, An exemplary embodiment of the present disclosure, an apparatus for inspecting a component, the apparatus comprising: a stage for supporting a component; an emitting unit configured to emit light to the component supported on the stage; a light receiving unit configured to receive transmitted light that has passed through the component or reflected light that has reflected from the component; a driving unit configured to relatively move the stage, the emitting unit, and the light receiving unit; and a controller, and the controller controls the emitting unit, the light receiving unit, and the driving unit to perform: an emitting operation of emitting light to the component; a light receiving operation of receiving the light from the component; and a predicting operation of predicting a fraction of fillers in the component through information about the received light, and the emitting operation, the light receiving operation, and the predicting operation are repeatedly performed while changing relative positions of the emitting unit, the light receiving unit, and the component, the information includes: a time taken to receive the light emitted in the emitting operation; a difference between an intensity of light emitted in the emitting operation and an intensity of light received in the light receiving operation; and a degree of refraction of the light emitted in the emitting operation, and the light emitted by the emitting unit may be far infrared ray.

According to the exemplary embodiment of the present invention, it is possible to inspect the distribution of fillers F in a component by a non-destructive inspection method.

Further, according to the exemplary embodiment of the present invention, it is possible to check the degree of dispersion of the fillers F from the manufacturing stage.

Effects of the present disclosure are not limited to those described above and effects not stated above will be clearly understood to those skilled in the art from the specification and the accompanying drawings.

### BRIEF DESCRIPTION OF THE DRAWINGS

The various features and advantages of the non-limiting exemplary embodiment of the present specification may become more apparent by reviewing the detailed description together with the accompanying drawings. The accompanying drawings are provided for illustrative purposes only and should not be construed as limiting the scope of claims. The accompanying drawings are not considered to be drawn to scale unless explicitly stated. For clarity, the various dimensions of the drawings may have been exaggerated.

FIG. 1 is a diagram schematically illustrating a component inspection apparatus according to an exemplary embodiment of the present invention.

FIG. 2 is a graph illustrating waveforms of light received by a light receiving unit according to fractions of fillers.

FIG. 3 is a diagram schematically illustrating an exemplary embodiment of an emitting unit and the light receiving unit of FIG. 1.

FIG. 4 is a diagram schematically illustrating another exemplary embodiment of the emitting unit and the light receiving unit of FIG. 1.

FIG. 5 is a diagram illustrating an appearance of a component inspection apparatus according to another exemplary embodiment of the present invention.

FIG. 6 is a flowchart illustrating a component inspection method according to an exemplary embodiment of the present invention.

FIG. 7 is a diagram illustrating a state in which light is continuously emitted to a plurality of regions while moving a component.

FIG. 8 is a diagram illustrating a state in which light is continuously emitted to a plurality of regions while rotating the component.

### DETAILED DESCRIPTION

Example embodiments will now be described more fully with reference to the accompanying drawings. Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

When the term “same” or “identical” is used in the description of example embodiments, it should be understood that some imprecisions may exist. Thus, when one element or value is referred to as being the same as another element or value, it should be understood that the element or value is the same as the other element or value within a manufacturing or operational tolerance range (e.g., ±10%).

When the terms “about” or “substantially” are used in connection with a numerical value, it should be understood that the associated numerical value includes a manufacturing or operational tolerance (e.g., ±10%) around the stated numerical value. Moreover, when the words “generally” and “substantially” are used in connection with a geometric shape, it should be understood that the precision of the geometric shape is not required but that latitude for the shape is within the scope of the

disclosure.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

Hereinafter, a component inspection apparatus and a component inspection method of the present invention will be described with reference to the accompanying drawings.

In the present exemplary embodiment to be described below, the present invention will be described based on the case where a component P to be inspected is a component P used in an apparatus for processing a substrate (wafer or glass substrate). According to an example, the component P may be an O-ring. However, the present invention is not limited thereto, and the technical spirit of the present invention may be applied to components used in other apparatuses.

Furthermore, the component P may be composed of a matrix M and a filler F. The matrix M may be made of a polymer and a plastic-based material. The filler F may be made of a metal or ceramic material. The filler F may have various shapes, such as a spherical shape, a square shape, a triangle, an oval shape, and a wire shape. Furthermore, the filler F may have a size of 1 nm to 50 um.

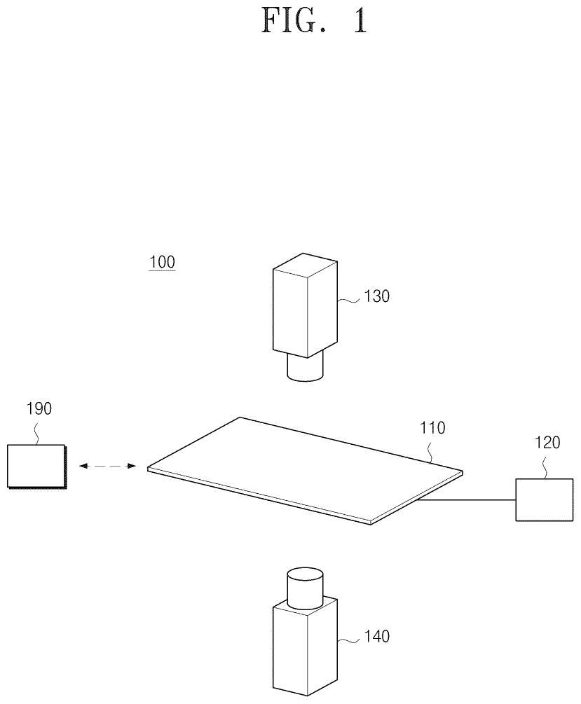

FIG. 1 is a diagram schematically illustrating a component inspection apparatus according to an exemplary embodiment of the present invention. Referring to FIG. 1, a component inspection apparatus 100 may include a stage 110, a driving unit 120, an emitting unit 130, a light receiving unit 140, and a controller 190.

The stage 110 is provided to support the component P to be inspected. The stage 110 is provided to fix the component P to be inspected. The shape of the stage 110 is not limited to one shape, and any structure capable of supporting the component P in consideration of the configuration and arrangement of the emitting unit 130 and the light receiving unit 140 and the shape and structure of the component P is sufficient. According to an example, the stage 110 is provided in a plate shape, and the component P may be placed on the stage. In addition, the stage 110 may include one or more fixing supports (e.g., tongs, grippers, or racks).

The driving unit 120 may relatively move the stage 110, the emitting unit 130, and the light receiving unit 140. Hereinafter, the present invention will be described based on the case of moving the stage 110 as an example.

The driving unit 120 is provided to move the stage 110. The driving unit 120 may be provided to move the stage 110 in a vertical or horizontal direction or rotate the stage 110. According to an example, the driving unit 120 may be a motor or a stepping motor. However, the present invention is not limited thereto, and the driving unit 120 may include a known component for driving the stage 110.

The emitting unit 130 emits light toward the component P. The emitting unit 130 may emit light to a first region, which is a part of the component P. Light may be provided in a wavelength of 2 um to 5 mm. According to an example, the light may be far infrared rays. Light having a wavelength less than the above-described wavelength range may heat the component P to cause deformation and damage, and light having a wavelength exceeding the above-described range may be scattered so that it may be difficult for the light receiving unit 140 to obtain accurate measured values.

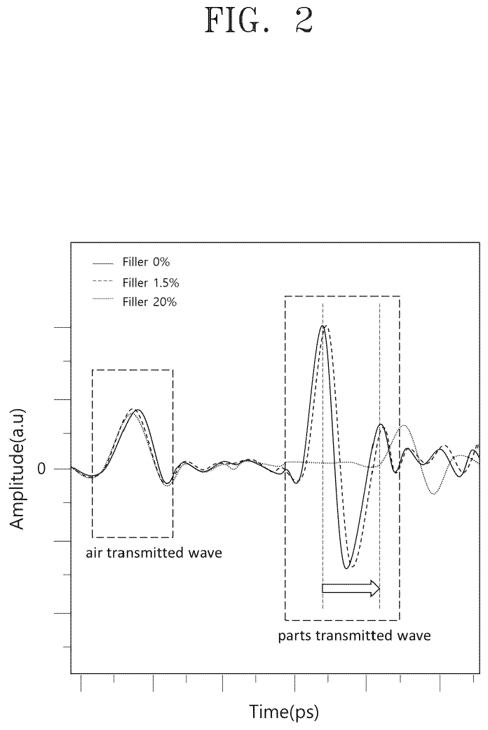

The light receiving unit 140 receives light from the component P. The light receiving unit 140 may be provided to transmit information included in the received light to the controller 190. FIG. 2 is a graph illustrating waveforms of light received by the light receiving unit according to fractions of fillers. Referring to FIG. 2, the information may be provided to obtain information, such as a wavelength and an intensity of received light, a time taken to receive light emitted by the emitting unit 130, and a refractive index.

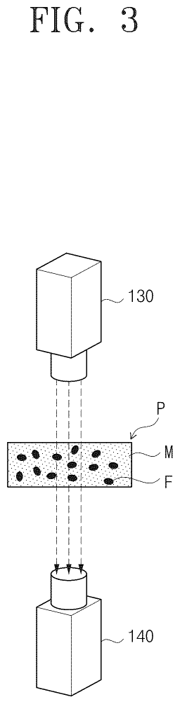

The light received by the light receiving unit has been emitted by the emitting unit 130, and may be transmitted light that has passed through the component P. FIG. 3 is a diagram schematically illustrating a state in which the light receiving unit receives transmitted light. Referring to FIG. 3, the light receiving unit 140 receives the transmitted light that has passed through the component P. The light receiving unit 140 may be positioned on an extension line of the light path emitted by the emitting unit 130 to receive the transmitted light. Accordingly, the emitting unit 130 and the light receiving unit 140 may be positioned to face each other with the component P interposed therebetween.

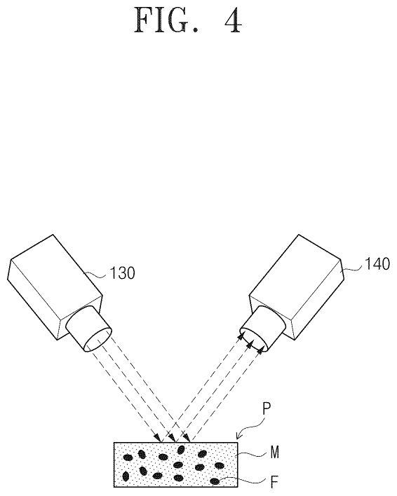

Also, the light received by the light receiving unit 140 has been emitted from the emitting unit 130, and may be reflected light reflected by the component P. FIG. 4 is a diagram schematically illustrating a state in which the light receiving unit receives reflected light. Referring to FIG. 4, the light receiving unit 140 receives reflected light reflected from the component P. The emitting unit 130 may obliquely emit light to the component P in order for the light receiving unit 140 to easily receive the reflected light. The light receiving unit 140 may be arranged to receive the reflected light reflected by the component P. The light receiving unit 140 may be electrically connected to the controller 190 and may transmit information in a wired or wireless manner. FIG. 4 is a diagram illustrating the component inspection apparatus according to another exemplary embodiment of the present invention. Referring to FIG. 4, a plurality of emitting units 130 and light receiving units 140 may be provided. A plurality of emitting units 130 and a plurality of light receiving units 140 may be provided to simultaneously emit light to the component and receive light. Accordingly, a plurality of regions A1-1 to A1-3, A2-1 to A2-3, and A3-1 to A3-3 of the component P may be simultaneously and continuously inspected.

The controller 190 may inspect the component P by controlling all operable components included in the component inspection apparatus 100. The controller 190 may effectively manage and execute the entire component inspection process, such as movement, position adjustment, and operation of the stage 110, the emitting unit 130, and the light receiving unit 140.

The controller 190 includes a process controller, a user interface, and a storage unit. The process controller is composed of a microprocessor (computer) and executes overall control of the component inspection apparatus. In this process, the process controller may receive data from components in the apparatus. The process controller may receive information by wire or wirelessly. Reference data and inspection recipe may be stored in a storage unit. The reference data may be reflected light or transmitted light data according to the fraction of the fillers F in the component P. The controller 190 may compare the reference data with the transmitted light or reflected light data received by the light receiving unit 140. The inspection recipe is a program for executing an inspection on each component according to various data and inspection conditions. According to an example, the inspection recipe may include a wavelength of light emitted from the emitting unit 130 and a method of moving the stage 110. The user interface and the storage unit are connected to the process controller. The processing recipe is stored in a storage medium in the storage unit, which may be a portable disk such as a hard disk, a CD-ROM, a DVD, or a semiconductor memory such as a flash memory. The user interface and the storage unit are connected to the process controller.

Hereinafter, a method of inspecting the component will be described. The component inspection method described below may be performed by the component inspection apparatus 100 described with reference to FIGS. 1 to 5. Accordingly, hereinafter, a component inspection method according to an exemplary embodiment will be described by referring to reference numerals illustrated in FIGS. 1 to 5 as they are. In addition, the component inspection method described below may be performed by controlling the components included in the component inspection apparatus 100 described above by the controller 130. The controller 190 may adjust the operation of each component according to the stored inspection recipe and inspect the component P from light received by the light receiving unit 140 through the stored data.

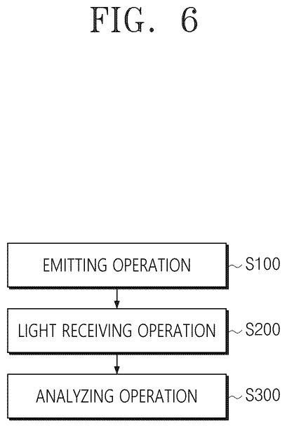

FIG. 6 is a flowchart illustrating a component inspection method according to an exemplary embodiment of the present invention. Referring to FIG. 6, the component inspection method may include an emitting operation S100, a light receiving operation S200, and an analyzing operation S300.

The emitting operation S100 is an operation of emitting light from the emitting unit 130 to the component P. In the emitting operation S100, the emitting unit 130 emits light to a first region of the component P. The first region may be a partial region of the component. The emitted light may be provided in a wavelength of 2 um to 5 mm. The light emitted by the emitting unit 130 may pass through the component P or may be reflected from the component P.

The light receiving operation S200 is an operation of receiving transmitted light that has passed through the component P or reflected light reflected from the component P. In the emitting operation S100, the light emitted to the first region of the component P is deformed and delayed according to the material and property of the first region of the component P and the like. The light reaches the light receiving unit 140 and is received by the light-receiving unit 140. Accordingly, the light received in the light receiving operation S200 may include information on the first region of the component P. The information may be expressed in terms of a wavelength and an intensity of the received light, a time taken to receive the light emitted by the emitting unit 130, a refractive index, and the like. The light receiving unit 140 transmits the information to the controller 190. Transmission to the controller 190 may be performed by a wireless or wired communication method.

In the analyzing operation S300, the fraction of the fillers F in the first region of the component may be measured from the light received by the light receiving unit 140. The controller 190 may measure the fraction of the fillers F in the first area by comparing the reference data previously stored in the controller 190 with the information received from the light receiving unit.

The reference data may be a set of information obtained in advance before performing the component inspection method. The reference data may be obtained through the following process.

A plurality of components P having different fractions of fillers for one component P is prepared. For each component P, the emitting unit 130 emits light and the light receiving unit 140 receives light. Information included in the received light is classified and stored according to the fraction of the fillers.

The information obtained by the above-described procedure may be stored in a storage unit of the controller 190. The controller 190 may compare the information obtained from the first region of the component P to be inspected with the above-described reference data to predict the fraction of the fillers F in the first region. As illustrated in FIG. 2, as the fraction of the fillers increases, the intensity of light received by the light receiving unit 140 decreases and the arrival time of the light to the light receiving unit 140 tends to be delayed.

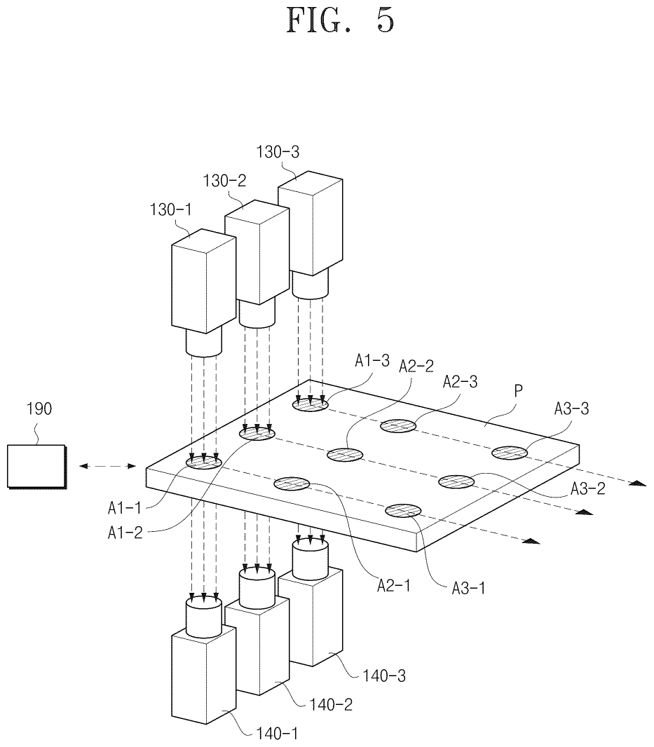

The component inspection method according to the exemplary embodiment of the present invention may be simultaneously performed on the plurality of regions A1-1, A1-2, and A1-3 of the component P, as illustrated in FIG. 5. The controller 190 may receive information from the plurality of light receiving units 140 and may predict the fraction of the fillers F for each region.

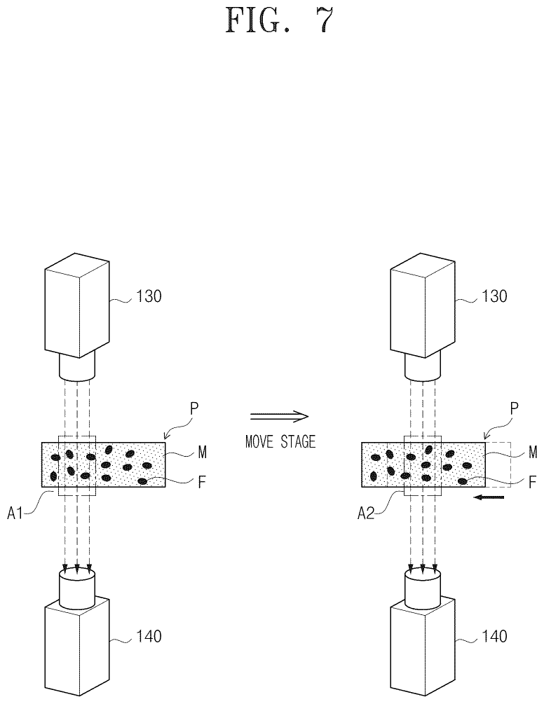

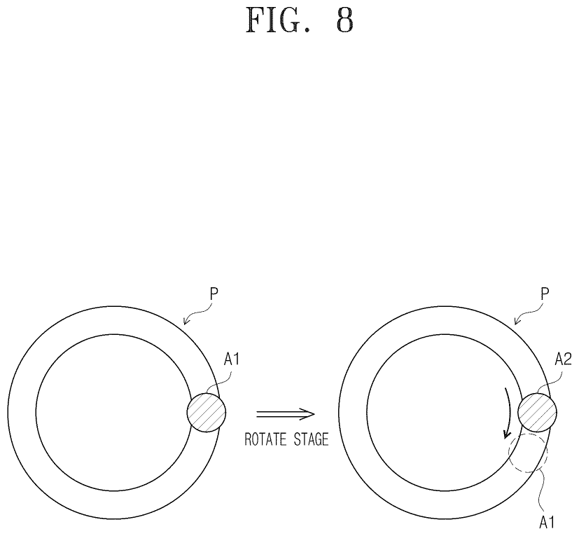

In addition, the component inspection method according to the exemplary embodiment of the present invention may be continuously performed. As illustrated in FIGS. 7 and 8, the component inspection method may be performed on the first region A1 of the component, and the component inspection method may be continuously performed on the second region A2 different from the first region A1 by moving the stage 110.

The controller 190 may track a movement direction of the stage 110 and map and visualize the fraction of the fillers F based on the information obtained in the plurality of regions. Accordingly, it is possible to determine whether the filler F is properly dispersed in the component P. For example, whether the difference between the fraction of the fillers F measured in the first region A1 and the fraction of the fillers F measured in the second region A2 is within an acceptable range may be checked to evaluate whether the filler F is properly dispersed in the first region A1 and the second region A2.

In addition, the controller 190 may be provided to build an artificial intelligence neural network by collecting and learning the acquired information. Through this, the accuracy of prediction of the fraction and dispersion of the filler F in the component may be improved.

According to the exemplary embodiment of the present invention, the degree of dispersion of the filler F in the component P may be inspected in a non-contact and non-destructive manner. Through this, the quality of the component P may be managed by quantifying the degree of dispersion of the filler F in the component P.

In addition, the component inspection method according to the exemplary embodiment of the present invention may be also performed during the manufacturing of the component P. The component inspection method according to the exemplary embodiment of the present invention is performed during the manufacturing of the component P, thereby detecting defects of the component in advance.

The foregoing detailed description illustrates the present invention. Further, the above content shows and describes the exemplary embodiment of the present invention, and the present invention may be used in various other combinations, modifications, and environments. That is, the foregoing content may be modified or corrected within the scope of the concept of the invention disclosed in the present specification, the scope equivalent to that of the invention, and/or the scope of the skill or knowledge in the art. The foregoing exemplary embodiment describes the best state for implementing the technical spirit of the present invention, and various changes required in specific application fields and uses of the present invention are possible. Accordingly, the detailed description of the invention above is not intended to limit the invention to the disclosed exemplary embodiment. Further, the accompanying claims should be construed to include other exemplary embodiments as well.

What is claimed is:

1. An apparatus for inspecting a component, the apparatus comprising:
a stage for supporting a component;
an emitting unit configured to emit light to the component supported on the stage;
a light receiving unit configured to receive light from the component supported on the stage; and
a controller, and
wherein the controller predicts a fraction of fillers in the component through information about the light received by the light receiving unit,
the information includes:
a time taken to receive the light emitted by the emitting unit;
a difference between an intensity of light emitted in the emitting unit and an intensity of light received in the light receiving unit; and
a degree of refraction of the light emitted in the emitting operation.

2. The apparatus of claim 1, wherein the light received by the light receiving unit is reflected light reflected from the component.

3. The apparatus of claim 1, wherein the light received by the light receiving unit is transmitted light that has passed through the component.
The apparatus of claim 1, wherein the light emitted by the emitting unit is far infrared ray.

5. The apparatus of claim 1, further comprising:
a driving unit configured to relatively move the stage, the emitting unit, and the light receiving unit,
wherein the controller controls the driving unit to emit the light to a first region of the component by the emitting unit and then continuously emit the light to a second region different from the first region.

6. The apparatus of claim 5, wherein the driving unit is provided to move the stage.

7. The apparatus of claim 6, wherein the emitting unit and the light receiving unit are provided in plural,
the plurality of emitting units is provided to emit light to different regions among the regions of the component,
each of the plurality of light receiving units is provided to receive the light emitted by the corresponding emitting unit, and
the controller controls the plurality of emitting units to emit light at the same time.

8. The apparatus of claim 1, wherein the filler is a metal material.

9. The apparatus of claim 8, wherein the filler is a spherical particle having a diameter of 1 nm to 50 um.

10. A method of inspecting a component, the method comprising:
an emitting operation of emitting light to the component;
a light receiving operation of receiving the light from the component; and
a predicting operation of predicting a fraction of fillers in the component through information about the received light,
wherein the information includes:
a time taken to receive the light emitted in the emitting operation;
a difference between an intensity of light emitted in the emitting operation and an intensity of light received in the light receiving operation; and
a degree of refraction of the light emitted in the emitting operation.

11. The method of claim 10, wherein the light received in the light receiving operation is reflected light reflected from the component.

12. The method of claim 10, wherein the light received by the light receiving operation is transmitted light that has passed through the component.

13. The method of claim 10, wherein the light emitted in the emitting operation is far infrared ray.

14. The method of claim 10, wherein the method is performed on a first region of the component and then continuously performed on a second region different from the first region.

15. The method of claim 14, wherein the method is repeated as the component is moved.

16. The method of claim 15, wherein the method is performed simultaneously on a plurality of regions different from the first region.

17. The method of claim 10, wherein the filler is a metal material.

18. An apparatus for inspecting a component, the apparatus comprising:
a stage for supporting a component;
an emitting unit configured to emit light to the component supported on the stage;
a light receiving unit configured to receive transmitted light that has passed through the component or reflected light that has reflected from the component;
a driving unit configured to relatively move the stage, the emitting unit, and the light receiving unit; and
a controller, and
the controller controls the emitting unit, the light receiving unit, and the driving unit to perform:
an emitting operation of emitting light to the component;
a light receiving operation of receiving the light from the component; and
a predicting operation of predicting a fraction of fillers in the component through information about the received light, and
the emitting operation, the light receiving operation, and the predicting operation are repeatedly performed while changing relative positions of the emitting unit, the light receiving unit, and the component,
the information includes:
a time taken to receive the light emitted in the emitting operation;
a difference between an intensity of light emitted in the emitting operation and an intensity of light received in the light receiving operation; and
a degree of refraction of the light emitted in the emitting operation, and
the light emitted by the emitting unit is far infrared ray.

19. The apparatus of claim 18, wherein the emitting unit and the light receiving unit are provided in plural,
the plurality of emitting units is provided to emit light to different regions among the regions of the component,
each of the plurality of light receiving units is provided to receive light emitted by the corresponding emitting unit, and
the controller controls the plurality of emitting units to emit light at the same time.

20. The apparatus of claim 19, wherein the filler is a metal material.
